(19) United States
(12) Patent Application Publication
(10) Pub. No.: US 2025/0137014 A1
(43) Pub. Date: May 1, 2025

(54) MANIPULATION OF ZONA PELLUCIDA TO FACILITATE DELIVERY OF NUCLEIC ACIDS FOR GENE EDITING IN EMBRYO

(71) Applicant: Colossal Biosciences Inc., Austin, TX (US)

(72) Inventors: Eriona Hysolli, Northfield, NH (US); Mariana Ianello Giassetti, Georgetown, TX (US)

(21) Appl. No.: 18/927,480

(22) Filed: Oct. 25, 2024

Related U.S. Application Data
(60) Provisional application No. 63/593,711, filed on Oct. 27, 2023.

(51) Int. Cl. C12N 15/873; C12N 5/073; C12N 9/26; C12N 15/86

(52) U.S. Cl. CPC C12N 15/873; C12N 5/0603; C12N 9/2474; C12N 15/86; C12N 2500/02; C12N 2523/00; C12N 2740/15043; C12N 2750/14143; C12Y 302/01035

(57) ABSTRACT

This disclosure relates to methods of transfecting or transducing mammalian zygotes with a nucleic acid of interest (such as e.g., ancient DNA). The methods generally include manipulating the zona pellucida of a denuded zygote to increase permeability of the zona pellucida to a nucleic acid of interest. In certain embodiments, the manipulating of the zona pellucida of the denuded zygote includes removing the zona pellucida with enzymatic treatment (with e.g., acid Tyrode, collagenase, hyaluronidase, or pronase). In other embodiments, the manipulating of the zona pellucida of the denuded zygote includes puncturing 3 to 5 holes in a zona pellucida of a denuded zygote using a laser; and contacting the nucleic acid of interest with the denuded zygote under conditions allowing transfection to generate a transfected denuded zygote. The nucleic acid of interest may be on a vector such as a recombinant viral vector.

### CROSS-REFERENCE TO RELATED APPLICATIONS

This application claims priority to U.S. Provisional Application No. 63/593,711, filed Oct. 27, 2023, the disclosure of which is herein incorporated by reference in its entirety.

### FIELD OF THE INVENTION

This disclosure generally relates to delivery of nucleic acids to a zygote by manipulating the permeability of the zona pellucida to allow passage of the nucleic acids. Specifically, this disclosure relates to using a laser or enzymatic treatment to weaken the zona pellucida to allow passage of the nucleic acids.

### BACKGROUND OF THE INVENTION

Lentiviruses (LENs) and Adeno-associated viruses (AAVs) have emerged as powerful vehicles for efficient gene delivery into embryos, in vivo and in vitro primary and immortalized cells. These vectors seamlessly integrate, and replicate provided genes within the host genome, making them indispensable tools for creating stable cell lines and generating transgenic animals. Concurrently, multiplex transduction in embryos has emerged as a technique that simultaneously introduces multiple genetic elements or genes into embryos using engineered viruses. This approach enables precise and efficient modifications to numerous genes within a single embryo, which is particularly beneficial in research scenarios focusing on intricate genetic interactions and creating customized embryos for various scientific objectives, including gene therapy, and developing animal models for further studies.

However, the zona pellucida, a protective barrier encompassing fertilized eggs, poses a formidable challenge to LENs and AAVs when attempting to transduce embryos. The zona's primary role in safeguarding early embryos from environmental threats, including viral infection, directly clashes with viral-based gene delivery. Prior efforts to chemically weaken or remove the zona have proved detrimental, significantly impairing embryonic development and maturation. The absence of the zona renders fertilized eggs vulnerable, adhesive, and prone to damage. Consequently, researchers have relied on single-cell embryo microinjection as a standard method for zona penetration, a technique that, while highly effective, demands skilled technologists and expensive microinjection workstations.

What is needed is an improved way to introduce nucleic acids of interest, e.g., via a vector containing the nucleic acid of interest, into an embryo (or zygote).

### SUMMARY OF THE INVENTION

The disclosure provides methods of transfecting or transducing mammalian zygotes with a nucleic acid of interest. The methods generally involve manipulating the zona pellucida of denuded zygotes to increase the permeability to nucleic acids of interest. The manipulating of the zona pellucida includes: (1) removal of the zona pellucida using enzymatic digestion; or (2) puncturing 3 to 5 holes in the zona pellucida. After manipulation, the methods include contacting the nucleic acid of interest with the denuded zygote under conditions allowing transfection to generate a transfected denuded zygote. In other words, the disclosure relates to methods of delivering nucleic acids of interest to denuded zygotes.

In some embodiments, the methods include puncturing 3 to 5 holes in a zona pellucida of a denuded zygote using a laser; and contacting the nucleic acid of interest with the denuded zygote under conditions allowing transfection to generate a transfected denuded zygote. In other embodiments, the methods include partially or completely removing the zona pellucida using enzymatic treatment (with e.g., acid Tyrode, collagenase, hyaluronidase, or pronase); and contacting the nucleic acid of interest with the denuded zygote under conditions allowing transfection to generate a transfected denuded zygote.

The methods of the disclosure may be used to transfect mammalian zygotes with any nucleic acid of interest. In some embodiments, the nucleic acid of interest is DNA or RNA. In one embodiment, the nucleic acid of interest is DNA, such as for example, ancient DNA.

In some embodiments, the zygote is denuded between 15 to 18 hours post-fertilization. The denuded zygote may be obtained via enzymatic treatment, such as e.g., treatment with hyaluronidase (e.g., 0.1 mg/ml).

The puncturing can be achieved using a pulsed laser. In certain embodiments, the laser pulse lasts for up to 800 μs, alternatively from about 650 μs to about 800 μs.

In some embodiments, the methods include further steps of culturing. For example, the methods can include culturing the zygote after transfection. The methods can also include culturing the cells after puncturing the holes and before contacting. For example, the methods include culturing the cells for one hour at a temperature of about 38.5° C. under an atmosphere of 5% CO₂ and 5% O₂ after puncturing the holes and before contacting.

The step of contact includes contacting the cells with a vector, such as, e.g., a plasmid, or a recombinant viral vector containing the nucleic acid of interest. In certain embodiments, the recombinant viral vector is a retrovirus, a recombinant rabies virus, a recombinant lentivirus, or a recombinant adeno-associated virus (AAV), containing the nucleic acid of interest. In some embodiments, the vector is a recombinant lentivirus containing the nucleic acid of interest. In other embodiments, the vector is a recombinant AAV containing the nucleic acid of interest. A variety of suitable amounts of the vector may be used. For example, the methods include contacting the denuded zygote with from about 1.75×10⁷ GC/ml to about 7×10⁹ GC/ml of the vector.

The step of contacting in certain embodiments also includes culturing the denuded zygote in the presence of the nucleic acid, such as, for example, for 3 to 7 days, alternatively up to 14 days. In some embodiments, the culturing includes a temperature of about 38.5° C. under an atmosphere of 5% CO₂ and 5% O₂.

The methods may also include culturing the transfected denuded zygote. For example, the transfected denuded zygote may be cultured at a temperature of about 38.5° C. under an atmosphere of 5% CO₂ and 5% O₂.

In other embodiments, the methods further include testing the transfected denuded zygote for the presence of the nucleic acid of interest.

In some embodiments, the puncturing includes observing the puncturing with a microscope. In other embodiments, the methods include generating the denuded zygote. In certain embodiments, the denuding includes treatment with an enzyme, such as, e.g., hyaluronidase.

The methods of the disclosure may also include maturing the transfected denuded zygote. For example, the transfected denuded zygote may be matured into an embryo and/or into a mammal.

Other features and advantages of the invention will be apparent from the detailed description and examples that follow.

### BRIEF DESCRIPTION OF THE DRAWINGS

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1 shows bovine zygotes before (FIG. 1A) and after (FIG. 1B) perforation of the zona pellucida (ZP) using a laser.

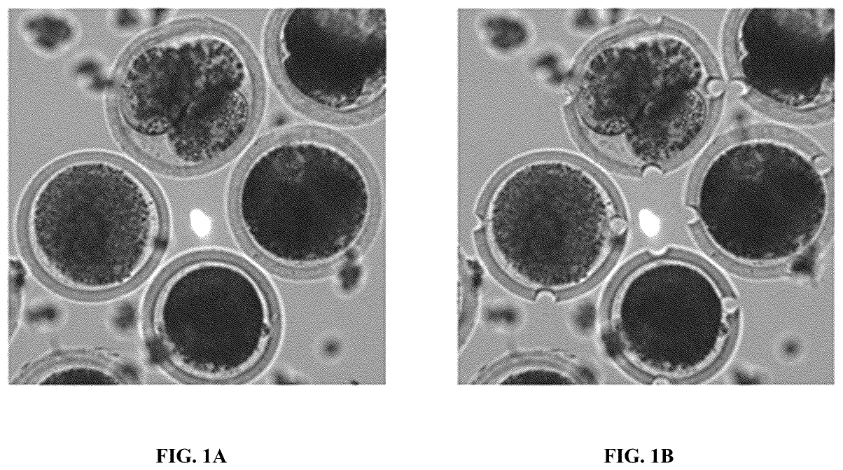

FIG. 2 shows representative images of bovine embryos seven days after fertilization from three different replicates and transduction conditions after ZP Perforation with laser. Conditions described at replicate 2 provided the best outcomes considering the viability of embryos and expression of fluorescent marker (GFP) using CMV_GFP_Poly A_AAV). The arrow indicates GFP+ blastocyst.

FIG. 3 shows representative images of bovine embryos seven days after fertilization co-expressing GFP and BFP after ZP Puncture and multiplex transfection. The arrow indicates GFP+ BFP+ viable morula (>16 cells).

### DETAILED DESCRIPTION

Developing multiplex transduction systems in non-model species is crucial for introducing multiple genetic elements or genes into embryos from domesticated animals to endangered species, aiming for new molecular tools for conservation and de-extinction. Subsequent studies will focus on embryo genotyping to facilitate multiplex gene editing and the establishment of embryonic cell lines with stable genetic modifications. Once a high percentage of desirable multiplex indels is achieved, transduced blastocysts with perturbed ZP will either be cryopreserved or transferred to surrogate females to generate gene-edited offspring. Without being bound by theory, it is important to note that microinjection into the embryo can be a bit harsh for multiplex editing. Virus transduction can be less toxic and more multiplexable.

Accordingly, this disclosure relates to improved methods for transfecting zygotes. Specifically, this disclosure is based on the discovery that partially or entirely removing the zona pellucida with enzymatic treatment allows for a high transfection rate. Furthermore, this disclosure is based on the discovery that using a laser to generate between 3 to 5 holes (e.g., less than 6 holes) in the zona pellucida allows for high transfection rate without jeopardizing zygote survival.

This disclosure is also based on the discovery that it is possible to manipulate a zona pellucida of a denuded zygote to increase the permeability of the zona pellucida to a nucleic acid of interest. The manipulation can be achieved by enzymatic removal of the zona pellucida or by using a laser to puncture holes in the zona pellucida.

Accordingly, this disclosure provides methods of transfecting or transducing (virus-mediated transfer of nucleic acids into cells) mammalian zygotes with a nucleic acid of interest which include manipulating the zona pellucida of a denuded zygote; and contacting the nucleic acid of interest with the denuded zygote under conditions allowing transfection to generate a transfected denuded zygote. The zona pellucida may be manipulated via a variety of ways. In certain embodiments, the zona pellucida is manipulated by puncturing holes with a laser. In other embodiments, the zona pellucida is manipulated by entirely removing the zona pellucida from the denuded zygote using enzymatic treatment (under conditions sufficient to remove the zona pellucida).

In certain embodiments, this disclosure provides methods of transfecting mammalian zygotes with a nucleic acid of interest which include puncturing 3 to 5 holes in a zona pellucida of a denuded zygote using a laser; and contacting the nucleic acid of interest with the denuded zygote under conditions allowing transfection to generate a transfected denuded zygote. In other embodiments, provides methods of transfecting mammalian zygotes with a nucleic acid of interest which include fully removing the zona pellucida of a denuded zygote via enzymatic treatment; and contacting the nucleic acid of interest with the denuded zygote under conditions allowing transfection to generate a transfected denuded zygote. In other embodiments, provides methods of transfecting mammalian zygotes with a nucleic acid of interest which include partially removing the zona pellucida of a denuded zygote via enzymatic treatment; and contacting the nucleic acid of interest with the denuded zygote under conditions allowing transfection to generate a transfected denuded zygote.

The methods also include generating the denuded zygote and subsequent maturation of the zygote into an embryo and/or mammal.

The general description and the following detailed description are exemplary and explanatory only and are not restrictive of the invention, as defined in the appended claims. Other aspects of the present invention will be apparent to those skilled in the art in view of the detailed description of the invention as provided herein.

### Definitions

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein, the term “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

It is also to be understood that the terminology used herein is for the purpose of describing particular embodiments only and is not intended to be limiting.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consisting of” excludes any element, step, or ingredient not specified in the claim element.

As used herein, the term “zygote” refers to a diploid cell resulting from the fusion of two haploid gamete, i.e., a single-cell stage embryo.

As used herein, the term “zona pellucida” or “ZP” refers to a specialized extracellular matrix that surrounds the plasma membrane of zygotes.

As used herein, the term “perivitelline space” refers to the space between the zona pellucida and the cell membrane of an oocyte, a zygote, or a fertilized ovum.

As used herein, the term “polar body” refers to a small haploid cell that is formed at the same time as an egg cell during meiosis, but generally does not have the ability to be fertilized.

As used herein, the term “embryo” refers to an initial stage of development of a multicellular organism. “Embryo” or “embryonic development” is the part of the life cycle that begins just after fertilization of the egg cell with the sperm cell. The resulting fusion of the egg cell and the sperm cell results in a single-celled zygote that undergoes many cell divisions, ultimately resulting in a multicellular organism implanted in the lining of a womb, which allows for gastrulation, neurulation, and organogenesis of the developing organism. An “embryo” can also refer to an unborn or unhatched offspring in the process of development.

As used herein, the term “cumulus cells” refers to a group of closely associated granulosa cells that surround the oocyte and participate in the processes of oocyte maturation and fertilization.

As used herein, the term “capacitation” refers to the activation process that prepares the sperm for fertilizing an oocyte. Capacitation can refer to the activation process that occurs in the female reproductive tract that prepares the sperm to fertilize an oocyte. Alternatively, capacitation can refer to an artificial activation process comprising incubating the sperm in a specific medium for activating the sperm to prepare the sperm for fertilizing the oocyte.

Before certain embodiments are described in greater detail, it is to be understood that this invention is not limited to certain embodiments described, as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Before certain embodiments are described in greater detail, it is to be understood that this invention is not limited to certain embodiments described, as such may, of course, vary. It is also to be understood that the terminology used herein is for the purpose of describing certain embodiments only, and is not intended to be limiting, since the scope of the present invention will be limited only by the appended claims.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

For clarity of disclosure, and not by way of limitation, the detailed description of the invention is divided into subsections that describe or illustrate certain features, embodiments, or applications of the present invention.

### Methods of Transfecting Denuded Zygotes

The disclosure generally provides for methods of transfecting mammalian zygotes which rely on manipulating the zona pellucida to allow for transfection of a nucleic acid of interest. In certain embodiments, the methods manipulate a zona pellucida of a denuded zygote to increase the permeability of the zona pellucida to the nucleic acid of interest. As herein, “transfect”, “transfection”, or “transfecting” includes any delivery of the nucleic acid of interest to the zygote including transduction (i.e., delivery of the nucleic acid with a viral vector).

The methods can manipulate the zona pellucida of a denuded zygote to increase the permeability of the zona pellucida to the nucleic acid of interest by using a laser to puncture holes in the zona pellucida or by removal of the zona pellucida with enzymatic treatment.

One embodiment of the disclosure is directed to methods of transfecting mammalian zygotes using enzymatic treatment to partially or entirely remove the zona pellucida thereby allowing for the delivery of the nucleic acid (e.g., DNA molecule) that is to be introduced into the zygote.

Another embodiment of the disclosure is directed to methods of transfecting mammalian zygotes using a laser to puncture a hole or a plurality of holes into zona pellucida thereby allowing for the delivery of the nucleic acid (e.g., DNA molecule) that is to be introduced into the zygote. Specifically, this disclosure is based on the discovery that using 3, 4, or 5 (i.e., less than 6) holes in the zona pellucida allows for optimal transfection without reducing zygote viability.

Without being bound by theory, utilizing a laser for drilling a hole in the zona pellucida, instead of utilizing chemical means to drill a hole, allows for a precise hole to be drilled with minimal to no damage to the zygote, which can allow for the production of more higher quality transfected embryos from the in vitro fertilization process. Additionally, the laser is easier to manipulate (i.e., aim, adjust strength, etc.) and is much faster than utilizing chemical means. The laser can drill a hole through the zona pellucida without the help of a micromanipulation set while utilizing chimerical means results in the need for a micromanipulation set for aiming.

The methods of transfecting mammalian zygotes with a nucleic acid of interest generally include: manipulating the zona pellucida of a denuded zygote to allow passage of the nucleic acid of interest into the zygote (i.e. increasing the permeability of the zona pellucida to the nucleic acid of interest); and contacting the nucleic acid of interest with the denuded zygote under conditions allowing transfection to generate a transfected denuded zygote.

The manipulating of the zona pellucida of the denuded zygote includes puncturing 3 to 5 holes in the zona pellucida of the denuded zygote using a laser. Alternatively, the manipulating of the zona pellucida of the denuded zygote includes partially removing the zona pellucida with enzymatic treatment (with e.g., acid Tyrode, collagenase, hyaluronidase, or pronase (e.g., 0.5% pronase)). Alternatively, the manipulating of the zona pellucida of the denuded zygote includes entirely removing the zona pellucida with enzymatic treatment (with e.g., acid Tyrode, collagenase, hyaluronidase, or pronase).

One embodiment of the disclosure is a method of transfecting a mammalian zygote with a nucleic acid of interest comprising: manipulating a zona pellucida of a denuded zygote to increase the permeability of the zona pellucida to the nucleic acid of interest; and contacting the nucleic acid of interest with the denuded zygote under conditions allowing transfection to generate a transfected denuded zygote. In some embodiment the method of transfecting a mammalian zygote with a nucleic acid of interest wherein the manipulating a zona pellucida of a denuded zygote to increase the permeability of the zona pellucida to the nucleic acid of interest includes puncturing 3 to 5 holes in a zona pellucida of a denuded zygote using a laser; and contacting the nucleic acid of interest with the denuded zygote under conditions allowing transfection to generate a transfected denuded zygote. In other embodiments, the method of transfecting a mammalian zygote with a nucleic acid of interest wherein the manipulating a zona pellucida of a denuded zygote to increase the permeability of the zona pellucida to the nucleic acid of interest includes removing the zona pellucida with enzymatic treatment; and contacting the nucleic acid of interest with the denuded zygote under conditions allowing transfection to generate a transfected denuded zygote.

In one embodiment of the disclosure, the method of transfecting mammalian zygotes with a nucleic acid of interest includes: entirely removing the zona pellucida of a denuded zygote with enzymatic treatment (with e.g., acid Tyrode, collagenase, hyaluronidase, or pronase); and contacting the nucleic acid of interest with the denuded zygote under conditions allowing transfection to generate a transfected denuded zygote. In an alternate embodiment of the disclosure, the method of transducing mammalian zygotes with a nucleic acid of interest includes: entirely removing the zona pellucida of a denuded zygote with enzymatic treatment (with e.g. acid Tyrode, collagenase, hyaluronidase, or pronase); and contacting the nucleic acid of interest with the denuded zygote under conditions allowing transduction to generate a transduced denuded zygote.

In another embodiment of the disclosure, the method of transfecting mammalian zygotes with a nucleic acid of interest includes: puncturing 3 to 5 holes in a zona pellucida of a denuded zygote using a laser; and contacting the nucleic acid of interest with the denuded zygote under conditions allowing transfection to generate a transfected denuded zygote. In yet another embodiment of the disclosure, the method of transducing mammalian zygotes with a nucleic acid of interest includes: puncturing 3 to 5 holes in a zona pellucida of a denuded zygote using a laser; and contacting the nucleic acid of interest with the denuded zygote under conditions allowing transduction to generate a transduced denuded zygote.

In the methods of the disclosure, the holes are punctured into the zona pellucida using a laser. Specifically, in certain embodiments, the holes are punched into the zona pellucida using a pulsed laser (or a laser pulse).

Depending on the laser used, the length of the pulse can vary. A suitable laser pulse length includes up to 800 μs, alternatively from about 650 μs to 800 μs, alternatively from about 700 μs to 800 μs. In certain embodiments, the laser has a wavelength of 1460 nm and an average power of 300 mW. In other embodiments, the laser has a wavelength of 1460 nm and about a 90 mW mean power. In some embodiment, 75%, 80%, 95%, 90%, 95%, 100%, from about 75% to about 100%, or from about 70% to about 100% of the average power or mean power are used. In alternate embodiments, the laser has a wavelength from about 1460 nm to about 1620 nm.

In certain embodiment, the laser is part of a microscope such that when the denuded zygotes are placed on the stage of the microscope the operation of the laser is visible. Accordingly, in some embodiments, the methods include placing the denuded zygotes on a microscope prior to puncturing the hole. In certain embodiments, using the microscope, the laser is aimed at a portion of the zona pellucida. When a microscope is used, the puncturing can also be observed with the microscope. In certain embodiments, when the microscope is used, the denuded zygotes are placed under oil.

The laser is placed in a suitably close proximity to the denuded zygote to allow for puncturing of the zona pellucida.

After treatment with the laser, the denuded zygotes may be washed with a suitable culture medium, such as, e.g., equilibrated culture medium.

The method of the disclosure can be used to transfect any nucleic acid of interest. In certain embodiments of the disclosure, the methods are used to transfect the denuded zygote is ancient DNA, e.g., DNA isolated from ancient specimens. In some embodiments, the transfecting includes use a reporter nucleic acid molecule, such as e.g., a nucleic acid encoding GFP.

A variety of vectors may be used to deliver the nucleic acid of interest. Suitable vectors include but are not limited to a plasmid or a recombinant viral vector. In certain embodiments, the recombinant viral vector is a retrovirus, a recombinant lentivirus, or a recombinant adeno-associated virus (AAV). As used herein, a suitable vector also includes a lipid nanoparticle (LNP) containing the nucleic acid of interest or a virus-like particle (VLP) containing the nucleic acid of interest.

In one embodiment, the vector is a recombinant lentivirus. In another embodiment, the vector is a recombinant AAV. When a recombinant viral vector is used, the denuded zygote may be contacted with from about 1.75×10⁷ GC/ml to about 7×10⁹ GC/ml of the vector.

The methods of the disclosure also include culturing the cells.

In one embodiment, the methods include comprise culturing the denuded zygotes after puncturing the holes and before contacting. For example, the denuded zygotes may be cultured for one hour at a temperature of about 38.5° C. under an atmosphere of 5% CO₂ and 5% O₂ after puncturing the holes and before contacting. A variety of media may be used for such culturing. For example, the denuded zygotes may be cultured in an equilibrated culture medium.

The step of contacting includes culturing the denuded zygote in the presence of the nucleic acid for a suitable amount of time. In certain embodiments, the denuded zygote are cultured in the presence of the nucleic acid for 3 to 7 days, alternatively at least 3 days, alternatively about 3 to 5 days, alternatively about 1 to 7 days, alternatively about 4 to 6 days, alternatively up to 14 days, alternatively about 3 to 14 days, alternatively about 7 to 14 days. The culturing may be conducted under a variety of conditions. For example, the culturing may include culturing at a temperature of about 38.5° C. under an atmosphere of 5% CO₂ and 5% O₂.

After culturing the denuded zygote in the presence of the nucleic acid for a suitable amount of time, the transfected denuded zygote may be washed.

In certain embodiments, the method also includes culturing the transfected denuded zygote. Again, this culturing may be carried out under a variety of different conditions. For example, the culturing includes culturing at a temperature of about 38.5° C. under an atmosphere of 5% CO₂ and 5% O₂.

The methods of the disclosure also include testing the transfected denuded zygote for the presence of the nucleic acid of interest.

In certain embodiments, the mammal is selected from a dog, a cat, a fox, a tiger, a lion, a cheetah, a leopard, a jaguar, a wolf, a goat, a sheep, an elephant, a rabbit, an opossum, a porcupine, a lemur, an otter, a sloth, a kangaroo, a wolverine, a cattle, a buffalo, a horse, a caribou, a deer, a camel, an elk, a llama, an ox, a moose, a bear, a panda, a koala, a chimpanzee, a gorilla, a monkey, a giraffe, a seal, a hippopotamus, a rhinoceros, and a human. The mammal can, for example, be a horse.

### Denuding of Zygotes

In certain embodiments, the disclosure also includes denuding the zygote. In some embodiments, the zygotes are denuded between 15 to 18 hours post-fertilization.

The zygotes may be denuded using a variety of commonly known techniques including enzymatic treatment, aspiration with a pipette and vortexing.

In certain embodiments, the zygotes are denuded using enzymatic treatment. In a particular embodiment, the zygotes are denuded by treatment with hyaluronidase, such as e.g., a 0.1 mg/ml of a hyaluronidase solution. Other digestive enzymes may be used in place of hyaluronidase such as, for example, collagenase, Tryp^{LE} Express, trypsin/ethylenediaminetetraacetic (EDTA), and thermolysin.

Accordingly, in certain embodiments, the disclosure provides for methods of transfecting mammalian zygotes with a nucleic acid of interest comprising: generating a denuded zygote; puncturing 3 to 5 holes in a zona pellucida of the denuded zygote using a laser; and contacting the nucleic acid of interest with the denuded zygote under conditions allowing transfection to generate a transfected denuded zygote. In certain embodiments, the step of denuding includes treatment with an enzyme, such as, e.g., hyaluronidase.

Furthermore, in other embodiments, the disclosure provides for methods of transfecting mammalian zygotes with a nucleic acid of interest comprising: generating a denuded zygote; removing the zona pellucida of the denuded zygote with enzymatic treatment (with e.g. acid Tyrode, collagenase, hyaluronidase, or pronase); and contacting the nucleic acid of interest with the denuded zygote under conditions allowing transfection to generate a transfected denuded zygote. In certain embodiments, the step of denuding includes treatment with an enzyme, such as, e.g., hyaluronidase.

### Transfected Zygote Maturation

In further embodiments, the methods of the disclosure of include maturation of the transfected zygote into an embryo or mammal. In certain embodiments, maturation comprises implanting the transfected zygote or an embryo generated from the zygote into an animal host.

In other embodiments, the methods include in vitro maturation of the zygote into an embryo.

Without further description, it is believed that one of ordinary skill in the art can, using the preceding description and the following illustrative examples, make and utilize the present invention and practice the claimed methods. The following working examples, therefore, specifically point out the preferred embodiments of the present invention and are not to be construed as limiting in any way the remainder of the disclosure.

### EXAMPLES

### Example 1: Transfection of Bovine Zygotes Using a Laser

### Methods

Zygote generation. Bovine zygotes were procured through in vitro fertilization (IVF) to establish the proof of concept. Specifically, bovine zygotes were obtained by in vitro fertilization on day 1 (˜14-18 hours post-onset fertilization); presumptive zygotes (pZyg) were denuded with 0.1 mg/mg hyaluronidase solution (Sigma; h4272; Sigma; St. Louis, MO) in a washing medium (Bo_wash; IVF Biosciences; Falmouth, United Kingdom) under vortex for 2 minutes. Denuded presumptive zygotes were selected, washed 3× in washing medium, and 3× with M199H+10% FBS-based medium (holding medium). Around 50 pZyg were transferred to 50 μl of Holding medium micro drops under oil (Oil; IVF Biosciences). Using the laser associated with the microscope 9 (a Hamilton Thorne XYRCOS DTS), the ZPs were punctured. After the laser procedure, the pZyg were washed 3× in an equilibrated culture medium (BO_IVC) and placed in 500 μl of equilibrated BO_IVC (38.5° C. 5% CO₂ 5% O₂) for recovery for 1 hour.

Transfection using a laser. A laser system coupled with a 20× magnification lens microscope (XYRCOS; Hamilton Thorne) was employed, and precise punctures were made in the zygotes' zona pellucida (ZP). Different laser pulses, intensity, and number of punctures per embryo were tested. Following the laser procedure, the zygotes were placed in a balanced culture medium (BO_IVC) and recovered for one hour at 38.5° C. under an atmosphere of 5% CO₂ and 5% O₂.

Culture After Transfection. After the end of the virus transduction, zygotes were washed 3× in 500 ml of washing medium for 5 minutes using a round bottom well dish. Washed zygotes were cultured in a fresh culture plate containing 500 μl of pre-equilibrated BO_IVC under oil and cultured for up to 14 days in a controlled atmosphere chamber (38.5° C. 5% CO₂ and 5% O₂).

Various laser settings and puncture numbers (ranging from one to six per embryo) were tested to standardize the technique, considering embryonic viability, as assessed by the cleavage rate three days post-fertilization. Viable embryos with punctured ZP were then cultured in the presence of viruses (CBE_FGF5_TagBFP_bGH_scAAV2 and CMV_GFP_Poly A_AAV) within a controlled atmosphere chamber set at 38.5° C. with 5% CO₂ and 5% O₂. Different combinations of virus concentrations (ranging from 1.75×10⁷ GC/ml to 7×10⁹ GC/ml) and incubation durations (3 and 7 days) were tested to optimize the transduction process while maintaining embryo viability. Upon completion of the virus transduction, the washed embryos were transferred to fresh pre-equilibrated BO_IVC medium, placed under oil, and cultured for up to 14 days within a controlled atmosphere chamber (maintaining conditions at 38.5° C., 5% CO₂, and 5% O₂). Embryo development and viability assessments were conducted on days 3, 7, and 12 post-fertilizations in conjunction with fluorescence detection using inverted microscopy.

### Results

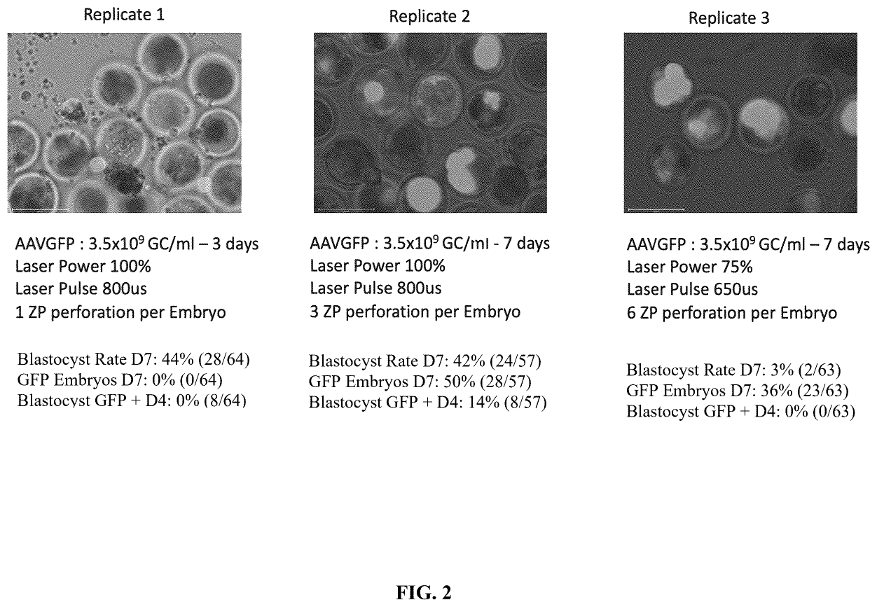

The results of the testing are summarized in Table 1-1 below. Table 1-2 describes samples tested in Table 1-1. Representative images of bovine embryos seven days after fertilization co-expressing GFP and BFP after ZP Puncture and multiplex transfection are shown in FIG. 2. The white arrow indicates GFP+ BFP+ viable morula (>16 cells).

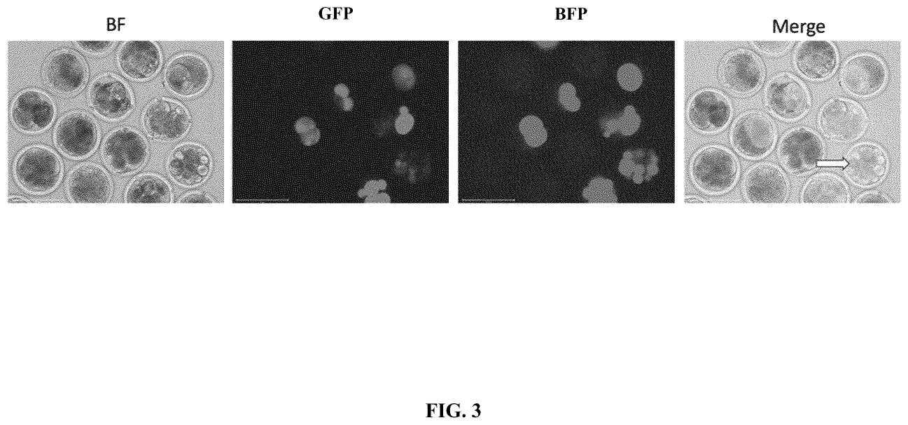

After four replicates of the studies utilizing more than ten different combinations in total between the number of perforations of ZP, laser power and pulse, diverse AAVs concentrations, and time of transduction, the condition of 3.5-7×10⁹ GC/ml, three perforations per embryo with laser at a power of 100% and pulse of 800 μs seems to be adequate to produce viable blastocysts of non-model species that are positive for the fluorescent markers (FIG. 2). The combination of >3.5×10° GC/ml of AAVs associated with more than six punctures per embryo was harmful, and no presence of GPF+ blastocyst was observed. However, the same AAV concentration with three holes of the ZP with an intense laser configuration provided 42% of blastocysts and 14% of GFP+ blastocysts. Using three laser punctures of ZP (power of 100% and 800 μs) appears not to affect embryo development at day 3 (cleavage rate) or day 7 (blastocyst rate) when compared to the control with no exposure to the virus and intact ZP (Table 1-1). The same previously described condition was used for the co-transduction CBE_FGF5_TagBFP_bGH_scAAV2 and CMV_GFP_Poly A_AAV, generating embryos co-expressing both fluorescent markers (see GFP and BFP; FIG. 3).

TABLE 1-1
Blastocyst and GFP+ rates of bovine embryos seven days after fertilization from
3 different replicates and transduction conditions after ZP Perforation with laser.

| | | | # ZP | | Blastocyst | Blastocyst |
|---|---|---|---|---|---|---|
| Sample | Virus | Concentration | perforation | Laser | rate % | GFP+ rate % |
| 1 | AAV2-CMV- | 3.5 × 10⁹ | yes 3 | power 100% & | 43 | 0 |
| | EGFP | 3 days | | pulse 800 μs | | |
| 2 | AAV2-CMV- | 3.5 × 10⁹ | yes 3 | power 100% & | 42 | 14 |
| | EGFP | 7 days | | pulse 800 μs | | |
| 3 | AAV2-CMV- | 3.5 × 10⁹ | yes 6 | power 75% & | 3 | 0 |
| | EGFP | 7 days | | pulse 650 μs | | |
| 4 | no | 0 | no | 0 | 54 | 0 |
| 5 | no | 0 | no | 0 | 60 | 0 |
| 6 | no | 0 | no | 0 | 26 | 0 |
| 7 | no | 0 | yes 3 | power 100% & | 42 | 0 |
| | | | | pulse 800 μs | | |
| 8 | no | 0 | yes 6 | power 75% & | 32 | 0 |
| | | | | pulse 650 μs | | |
| 9 | no | 0 | yes 3 | power 100% & | 53 | 0 |
| | | | | pulse 800 μs | | |

TABLE 1-2
Description of Samples used in Table 1-1

| Sample # | Sample Description |
|---|---|
| 1 | AAVGFP1 |
| 2 | AAVGFP2 |
| 3 | AAVGFP3 |
| 4 | Control 1 (no laser) |
| 5 | Control 2 (no laser) |
| 6 | Control 3 (no laser) |
| 7 | Control 1 (laser) |
| 8 | Control 2 (laser) |
| 9 | Control 3 (laser) |

While the invention has been described and illustrated herein by references to various specific materials, procedures, and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

### Embodiments

The invention provides also the following non-limiting embodiments.

Embodiment 1. A method of transfecting a mammalian zygote with a nucleic acid of interest comprising: manipulating a zona pellucida of a denuded zygote to increase the permeability of the zona pellucida to the nucleic acid of interest; and contacting the nucleic acid of interest with the denuded zygote under conditions allowing transfection to generate a transfected denuded zygote. The embodiments of the disclosure also encompass a method of transducing a mammalian zygote with a nucleic acid of interest comprising: manipulating a zona pellucida of a denuded zygote to increase the permeability of the zona pellucida to the nucleic acid of interest; and contacting the nucleic acid of interest with the denuded zygote under conditions allowing transduction to generate a transfected denuded zygote.

Embodiment 2. The method of embodiment 1, wherein the manipulating a zona pellucida of a denuded zygote to increase the permeability of the zona pellucida to the nucleic acid of interest comprises puncturing 3 to 5 holes in a zona pellucida of a denuded zygote using a laser.

Embodiment 3. The method of embodiment 1, wherein the manipulating a zona pellucida of a denuded zygote to increase the permeability of the zona pellucida to the nucleic acid of interest comprises removing the zona pellucida with enzymatic treatment. The removal with enzymatic treatment may remove the entire zona pellucida. In other embodiments, the removal with enzymatic treatment does not entirely remove all the zona pellucida.

Embodiment 4. The method of embodiment 3, wherein the enzymatic treatment comprises treatment with acid Tyrode, collagenase, hyaluronidase, or pronase.

Embodiment 5. The method of any one of embodiments 1 to 5, wherein the zygote is denuded between 15 to 18 hours post-fertilization.

Embodiment 6. The method of embodiment 5, wherein the denuding comprises enzymatic treatment.

Embodiment 7. The method of embodiment 6, wherein the enzymatic treatment comprises treatment with hyaluronidase.

Embodiment 8. The method of embodiment 7, wherein the enzymatic treatment comprises treatment with about 0.1 mg/ml of a hyaluronidase solution.

Embodiment 9. The method of embodiment 2, wherein the puncturing comprises use of a pulsed laser or laser pulse.

Embodiment 10. The method of embodiment 9, wherein the pulsed laser has a pulse of up to 800 μs.

Embodiment 11. The method of embodiment 9, wherein the pulse is about 650 μs to about 800 μs.

Embodiment 12. The method of any one of embodiments 1 to 11, further comprising culturing the zygote after transfection.

Embodiment 13. The method of any one of embodiments 1 to 12, wherein the method further comprises culturing the denuded zytgote for one hour at a temperature of about 38.5° C. under an atmosphere of 5% CO₂ and 5% O₂ after puncturing the holes and before contacting.

Embodiment 14. The method of any one of embodiments 1 to 13, wherein the contacting comprises contacting the cells with a vector comprising the nucleic acid of interest.

Embodiment 15. The method of embodiment 14, wherein the vector is a plasmid or a recombinant viral vector.

Embodiment 16. The method of embodiment 15, wherein the recombinant viral vector is a recombinant lentivirus.

Embodiment 17. The method of embodiment 15, wherein the recombinant viral vector is a recombinant AAV.

Embodiment 18. The method of any one of embodiments 15 to 17, wherein the method comprises contacting the denuded zygote with from about 1.75×10⁷ GC/ml to about 7×10⁹ GC/ml of the vector.

Embodiment 19. The method of any one of embodiments 1 to 18, wherein the contacting comprises culturing the denuded zygote in the presence of the nucleic acid.

Embodiment 20. The method of embodiment 19, wherein the contacting comprises culturing for 3 to 7 days.

Embodiment 21. The method of embodiment 20, wherein the culturing comprises culturing at a temperature of about 38.5° C. under an atmosphere of 5% CO₂ and 5% O₂.

Embodiment 22. The method of any one of embodiments 1 to 21, wherein the method further comprises culturing the transfected denuded zygote.

Embodiment 23. The method of embodiment 22, wherein the culturing comprises culturing at a temperature of about 38.5° C. under an atmosphere of 5% CO₂ and 5% O₂.

Embodiment 24. The method of any one of embodiments 1 to 24, wherein the method further comprises testing the transfected denuded zygote for the presence of the nucleic acid of interest.

Embodiment 25. The method of any one of embodiments 2 or 4 to 24, wherein the puncturing comprises observing the puncturing with a microscope.

Embodiment 26. The method of any one of embodiments 1 to 25, further comprising maturing the transfected denuded zygote into an embryo.

Embodiment 27. The method of embodiment 26, further comprising maturing the embryo into a mammal.

Embodiment 28. The method of any one of embodiments 1 to 25, wherein the nucleic acid of interest is DNA or RNA.

Embodiment 29. The method of embodiment 28, wherein the nucleic acid of interest is ancient DNA.

Embodiment 30. The method of any one of embodiments 1 to 29, further comprising denuding a zygote to generate the denuded zygote.

Embodiment 31. The method of embodiment 30, wherein the denuding comprises treatment with an enzyme.

Embodiment 32. The method of embodiment 31, wherein the enzyme is hyaluronidase.

What is claimed is:

1. A method of transfecting a mammalian zygote with a nucleic acid of interest comprising:
(a) manipulating a zona pellucida of a denuded zygote to increase the permeability of the zona pellucida to the nucleic acid of interest; and
(b) contacting the nucleic acid of interest with the denuded zygote under conditions allowing transfection to generate a transfected denuded zygote.

2. The method of claim 1, wherein the manipulating a zona pellucida of a denuded zygote to increase the permeability of the zona pellucida to the nucleic acid of interest comprises puncturing 3 to 5 holes in a zona pellucida of a denuded zygote using a laser.

3. The method of claim 1, wherein the manipulating a zona pellucida of a denuded zygote to increase the permeability of the zona pellucida to the nucleic acid of interest comprises removing the zona pellucida with enzymatic treatment.

4. The method of claim 3, wherein the enzymatic treatment comprises treatment with acid Tyrode, collagenase, hyaluronidase, or pronase.

5. The method of claim 1, wherein the zygote is denuded between 15 to 18 hours post-fertilization.

6. The method of claim 2, wherein the puncturing comprises use of a pulsed laser or laser pulse.

7. The method of claim 6, wherein the pulse is about 650 μs to about 800 μs or up to 800 μs.

8. The method of claim 1, further comprising culturing the zygote after transfection.

9. The method of claim 1, wherein the method further comprises culturing the denuded zygote for one hour at a temperature of about 38.5° C. under an atmosphere of 5% CO₂ and 5% O₂ after puncturing the holes and before contacting.

10. The method of claim 1, wherein the contacting comprises contacting the cells with a vector comprising the nucleic acid of interest.

11. The method of claim 10, wherein the vector is a plasmid or a recombinant viral vector.

12. The method of claim 11, wherein the recombinant viral vector is a lentivirus.

13. The method of claim 11, wherein the recombinant viral vector is a recombinant AAV.

14. The method of claim 1, wherein the contacting comprises culturing the denuded zygote in the presence of the nucleic acid.

15. The method of claim 14, wherein the contacting comprises culturing for 3 to 7 days.

16. The method of claim 1, wherein the method further comprises: culturing the transfected denuded zygote; testing the transfected denuded zygote for the presence of the nucleic acid of interest; maturing the transfected denuded zygote into an embryo; and/or maturing the transfected denuded zygote into an embryo and maturing the embryo into a mammal.

17. The method of claim 16, wherein the culturing comprises culturing at a temperature of about 38.5° C. under an atmosphere of 5% CO₂ and 5% O₂.

18. The method of claim 1, wherein the nucleic acid of interest is DNA or RNA.

19. The method of claim 18, wherein the nucleic acid of interest is ancient DNA.

20. The method of claim 1, further comprising denuding a zygote to generate the denuded zygote.

21. The method of claim 20, wherein the denuding comprises treatment with an enzyme.

22. The method of claim 21, wherein the enzyme is hyaluronidase.
